(19) United States
(12) Patent Application Publication
(10) Pub. No.: US 2009/0287081 A1
(43) Pub. Date: Nov. 19, 2009

(54) SUBMUCOSAL FIBROID ABLATION FOR THE TREATMENT OF MENORRHAGIA

(75) Inventors: Jessica GROSSMAN, San Francisco, CA (US); Teodoro BELLEZA, LaSelva Beach, CA (US); Graig GERBI, Half Moon Bay, CA (US); Michael MUNROW, Belmont, CA (US); Jordan BAJOR, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Gynesonics , Inc, Redwood City, CA (US)

(21) Appl. No.: 12/424,357

(22) Filed: Apr. 15, 2009

Related U.S. Application Data
(60) Provisional application No. 61/048,637, filed on Apr. 29, 2008.

(51) Int. Cl. A61B 18/18; A61B 8/00

(52) U.S. Cl. 600/439; 606/33; 600/407

(57) ABSTRACT

Submucosal fibroids are selectively treated by introducing a visualization and ablation instrument into a patient's uterus. Many or all of the fibroids are visualized, and the submucosal fibroids are identified. The identified submucosal fibroids are then selectively targeted, typically using a needle on the visualization and ablation instrument. In this way, the patients can be treated for menorrhagia without prolonging the time to treat intramural and subserosal fibroids which typically aren't responsible for menorrhagia.

### CROSS-REFERENCE TO RELATED APPLICATION

The present application claims the benefit of U.S. provisional patent application No. 61/048,637 (Attorney Docket No. 025676-001200US) filed on Apr. 29, 2008, the full disclosure of which is incorporated herein by reference.

### BACKGROUND OF THE INVENTION

1. Field of the Invention

The present invention relates generally to medical methods for the treatment of gynecological disorders. More particularly, the invention relates to a method for the selective ablation of submucosal fibroids for the treatment of menorrhagia.

Treatment of dysfunctional uterine bleeding remains an unmet clinical need. Fibroids are benign tumors of the uterine myometria (muscle) and are the most common tumor of the female pelvis. Fibroid tumors affect up to 30% of women of childbearing age and can cause significant symptoms such as discomfort, pelvic pain, menorrhagia, pressure, anemia, compression, infertility, and miscarriage. Fibroids may be located in the myometrium (intramural), adjacent the endometrium (submucosal), or in the outer layer of the uterus (subserosal).

Current treatments for fibroids include either or both pharmacological therapies and surgical interventions. Pharmacological treatments include the administration of medications such as NSAIDS, estrogen-progesterone combinations, and GnRH analogues. All medications are relatively ineffective and are palliative rather than curative. Surgical interventions include hysterectomy (surgical removal of the uterus) and myomectomy. Surgical myomectomy, in which fibroids are removed, is an open surgical procedure requiring laparotomy and general anesthesia. Often these surgical procedures are associated with the typical surgical risks and complications along with significant blood loss and can only remove a portion of the culprit tissue.

To overcome at least some of the problems associated with open surgical procedures, laparoscopic myomectomy was pioneered in the early 1990's. However, laparoscopic myomectomy remains technically challenging, requiring laparoscopic suturing, limiting its performance to only the most skilled of laparoscopic gynecologists. Other minimally invasive treatments for uterine fibroids include hysteroscopy and myolysis.

Hysteroscopy is the process by which a thin fiber optic camera is used to image inside the uterus and an attachment may be used to destroy tissue. Hysteroscopic resection is a surgical technique that uses a variety of devices (loops, roller balls, bipolar electrodes) to ablate or resect uterine tissue. The procedure requires the filling of the uterus with fluid for better viewing, and thus has potential side effects of fluid overload. Hysteroscopic ablation is limited by its visualization technique and thus, only appropriate for fibroids which are submucosal and/or protrude into the uterine cavity.

Myolysis was first performed in the 1980's using lasers or radio frequency (RF) energy to coagulate tissue, denature proteins, and necrose myometrium using laparoscopic visualization. Laparoscopic myolysis can be an alternative to myomectomy, as the fibroids are coagulated and then undergo coagulative necrosis resulting in a dramatic decrease in size. As with all laparoscopic techniques, myolysis treatment is limited by the fact that it can only allow for visualization of subserosal fibroids.

As an improvement over both hysteroscopy and needle myolysis, needle-based radiofrequency ablation under direct ultrasonic visualization has been proposed. Such procedures are described, for example in commonly-owned published PCT application PCT/US2007/066235, filed on Apr. 9, 2007, which claimed priority from application Ser. No. 11/564,164, filed on Nov. 28, 2006, and application Ser. No. 11/409,496, filed on Apr. 20, 2006, the full disclosures of which are incorporated herein by reference. In these applications, the identification and treatment of all fibroids in the uterine wall of a patient are described. An instrument including an ultrasonic transducer and an advanceable needle are introduced into the patient's uterus via a transvaginal approach. The ultrasonic transducer is used to identify fibroids, and the fibroids are sequentially treated by advancing the needles into the fibroid, applying radiofrequency energy, and optionally confirming that the entire volume of the fibroid has been treated.

While effective and valuable for many patients, the methods described in the above listed applications can be relatively long, particularly in patients having two or more fibroids. Moreover, for many patients, a primary if not sole complaint is excessive uterine bleeding, referred to as menorrhagia. In those patients, the menorrhagia is believed to be caused primarily by submucosal fibroids, and other fibroids, including intramural fibroids and subserosal fibroids are often asymptomatic.

For these reasons, it would be desirable to provide improved methods and protocols for treating fibroids in patients suffering from menorrhagia. In particular, it would be desirable to provide treatments which are effective for treating menorrhagia but which are office-based, less painful, relatively short and can avoid treating asymptomatic fibroids or otherwise overtreating the patient. At least some of these objectives will be met by the inventions described hereinbelow.

2. Brief Description of the Background Art

The commonly-owned applications referenced above have been published as U.S. 2007/0249936; U.S. 2007/0249939; and WO 2007/24265. It is noted that the methods described hereinafter may be performed using the apparatus described in these commonly-owned published applications, the full disclosures of which are incorporated herein by reference.

### BRIEF SUMMARY OF THE INVENTION

The present invention provides methods for ablating fibroids in women suffering from menorrhagia as a principal symptom and complaint. Menorrhagia is defined as excessive uterine bleeding occurring during menstruation. The bleeding commences at the expected time during the menstrual cycle, but is heavier than usual and often lasts longer than normal. Menorrhagia may be caused by a variety of disorders, including hormone imbalance, endometriosis, cancer, and, of particular interest to the present invention, the presence of uterine fibroids.

Uterine fibroids fall into several classifications, including submucosal (located at the uterine wall), intramural (located within the myometrium of the uterine wall), subserosal (located on or near the outside wall of the uterus), intracavitary fibroids (located almost or entirely within the uterine cavity), and pedunculated (located entirely outside of the uterine cavity and its walls). Of these, the submucosal fibroids are principally responsible for excessive uterine bleeding and menorrhagia. The intramural and subserosal myomas may cause other symptoms and problems, such as severe cramping, compression of adjacent organs, and the like, but are usually not responsible for excessive bleeding.

Thus, in accordance with the principles of the present invention, patients whose principal complaint is menorrhagia or excessive uterine bleeding may be treated by distinguishing between submucosal fibroids and all other fibroids (including intramural, subserosal, and intracavitary fibroids) and treating only the submucosal fibroids. In some cases, it may be possible to further selectively treat only those submucosal fibroids which have been determined to be responsible for the excessive bleeding, but in most cases it will be desirable to selectively treat all submucosal fibroids, and usually no other fibroids.

Patients suffering from menorrhagia or excessive uterine bleeding as a principal disorder or complaint will be those experiencing such excessive bleeding but who are generally free from other complaints associated with fibroids, such as cramping, compression of adjacent organs, and the like. For those patients suffering only from menorrhagia, the treatment protocols of the present invention may be shortened relative to protocols where all fibroids are treated, such as is suggested in the commonly-owned prior patent applications identified above.

Thus, the present invention provides methods for treating menorrhagia. The methods comprise providing a visualization and ablation instrument including a shaft having a distal end, an imaging transducer near the distal end of the shaft, and an energy delivery element near the distal end of the shaft. Exemplary devices suitable for use in the methods of the present invention are described in each of the commonly owned applications described and incorporated herein by reference above. The distal end of the shaft is advanced into the patient's uterus, and fibroids are visualized with the imaging transducer while the distal end of the shaft remains in the uterus. Submucosal fibroids are visualized and distinguished from all other fibroids, including at least intramural fibroids and subserosal fibroids, and the identified submucosal fibroids are selectively treated by delivering energy from the energy delivery element to the submucosal fibroid(s) while not targeting the other fibroid(s), if any.

### BRIEF DESCRIPTION OF THE DRAWINGS

FIG. 1A illustrates a visualization and ablation system embodying features of the present invention.

FIG. 1B illustrates features of an exemplary ultrasound probe of the visualization and ablation system of FIG. 1A.

FIG. 1C illustrates features of an exemplary ultrasound system of the visualization and ablation system of FIG. 1A.

FIG. 1D illustrates features of an exemplary radio frequency energy generator of the visualization and ablation system of FIG. 1A.

FIG. 1E illustrates the visualization and ablation system of FIG. 1A as disposed during operation within a uterus for the treatment of fibroids in accordance with the features of the present invention.

FIGS. 2A-2E illustrate use of the ultrasound visualization and ablation system of FIGS. 1A-1E for selectively ablating submucosal fibromas in accordance with the principles of the present invention.

### DETAILED DESCRIPTION OF THE INVENTION

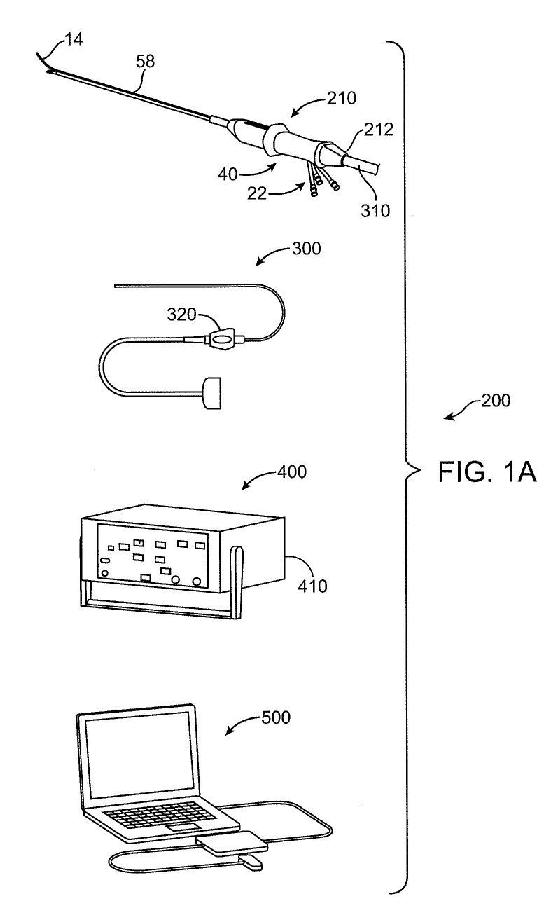

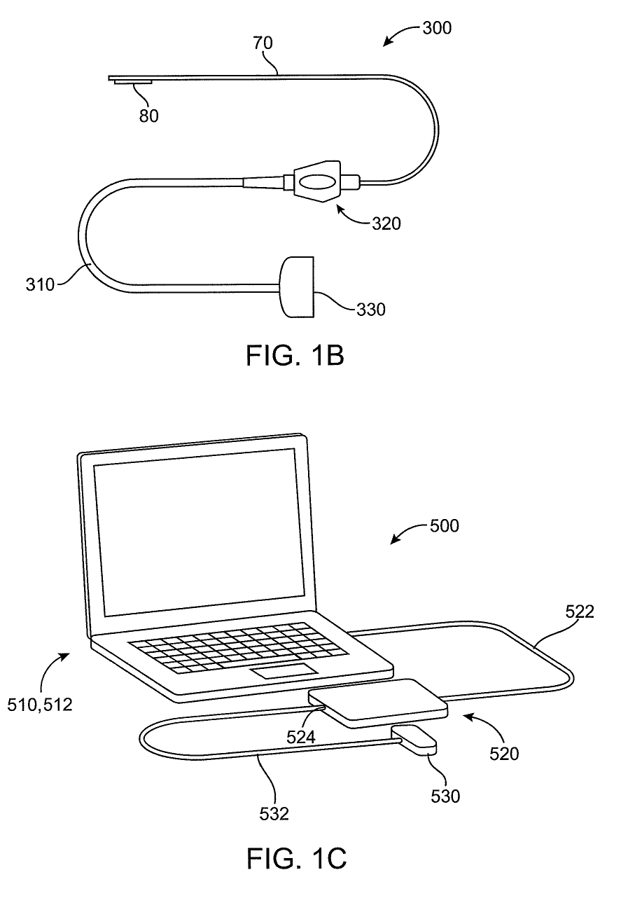

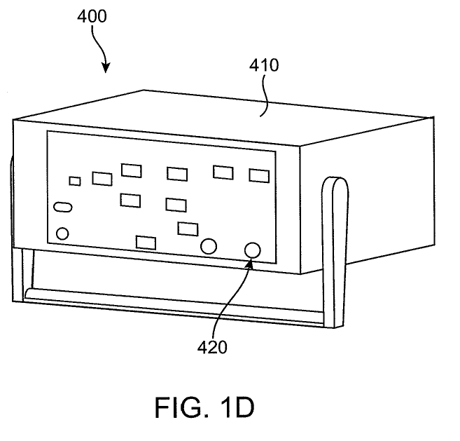

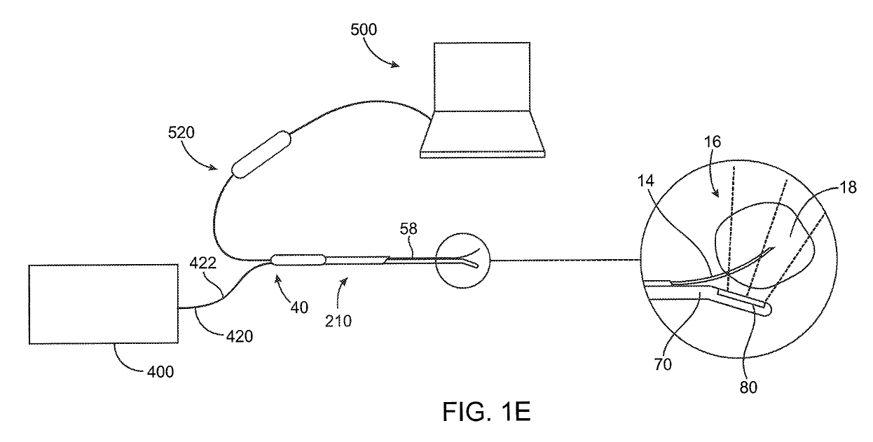

Referring to FIG. 1A, a visualization and ablation system 200 suitable for use in the methods of the present invention is shown, including a delivery device 210, an ultrasound imaging probe 300 detachable from the delivery device 210, a radio frequency energy generator 400, and an ultrasound system 500. The various components of the exemplary visualization and ablation system 200 will be further described in individual detail. The visualization and ablation system 200 is described in more detail in copending application PCT/US2007/066235, the full disclosure of which has previously been incorporated herein by reference. Other systems which may be utilized in performing the methods of the present invention are also described in PCT/US2007/066235, as well as in other commonly-owned, copending applications, including 11/347,018; 11/775,452; 12/198,861; and 61/156,270, the full disclosures of which are incorporated herein by reference.

FIGS. 1A-1E illustrate a system 200 for selectively delivering radiofrequency to ablate submucosal fibroids according to the methods of the present invention. This system is described in more detail in commonly owned PCT/US2007/066235. An ultrasound probe 300 embodying features of the present invention, is shown in FIG. 1B and generally includes an imaging insert 70 which includes transducer 80 (as shown in FIG. 1B) and is received in a the delivery device 210 through a port 212 at a proximal end 22 of the device 210. The ultrasound probe 300 includes an alignment element 320 for removably engaging with the port 212 of the delivery device 210 via a probe cable 310. Alignment element 320 is connectable to the ultrasound system 500 (FIG. 1C) by way of an ultrasound probe attachment element 330.

An ultrasound system controller 500, embodying features of the present invention, as shown in FIG. 1C, generally includes a CPU 510 such as one shown operable by a laptop computer 512. The CPU 510 is connectable to a beam former 520 by way of a communications cable (such as a firewire cable) such as an ultrasound cable 522. The beam former 520 is connectable at its distal end 524 to a probe attachment element 530 by a probe extension cable 532.

The visualization system is shown to be internal, i.e., utilizing an imaging sensor, such as an ultrasonic transducer, which is positionable within the uterus or other body cavity adjacent to the fibroid. Alternatively, external imaging from the patient's skin or from another body cavity could also be performed to assist in positioning the treatment element.

A radio frequency energy generator 400 includes an enclosure 410, embodying features of the present invention, is shown in FIGS. 1D and 1E and is generally connectable to the delivery device 210, including needle 14, through energy outlet port 420. A suitable cable 422 removably connects energy outlet port 420 to a needle port at the proximal end 22 of the handle 40. Radiofrequency energy is delivered from the radio frequency generator 400 to fibroid 18 at the target site 16 through needle 14 which is disposed within the needle guide 58.

As described above, the energy ablation source is radiofrequency energy. It will be appreciated that a wide variety of other sources could also be utilized, including optical or laser energy, microwave energy, thermal energy (more specifically cooling, cryoablation, or vapor), and the like.

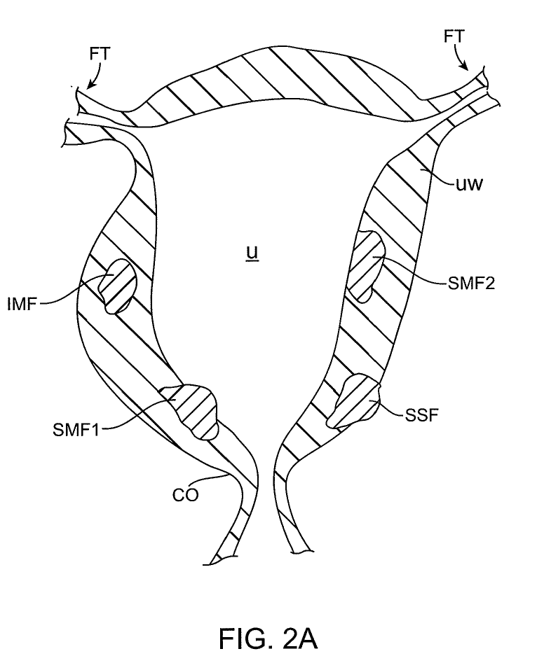

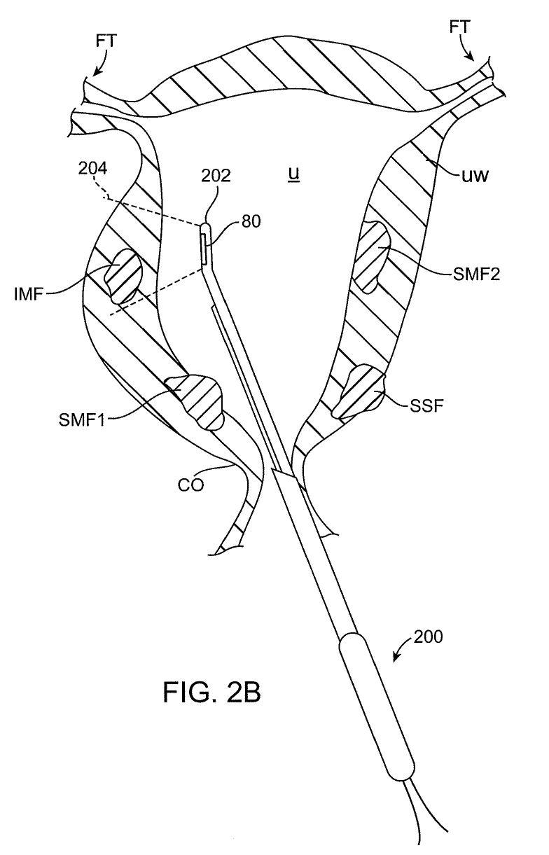

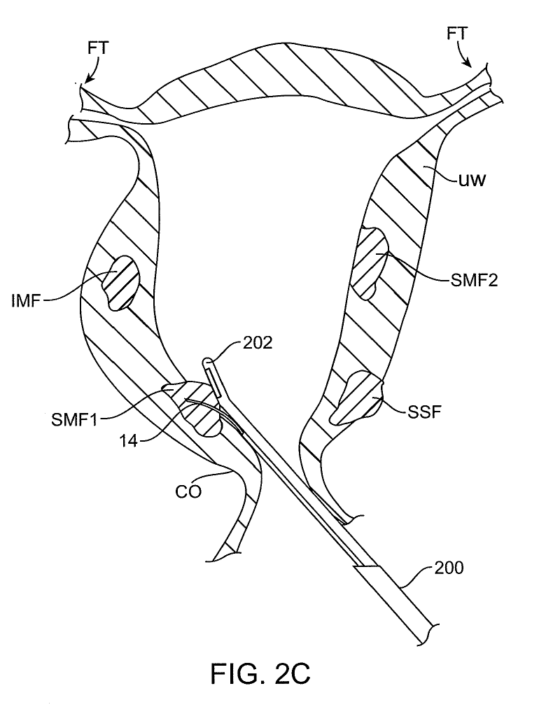

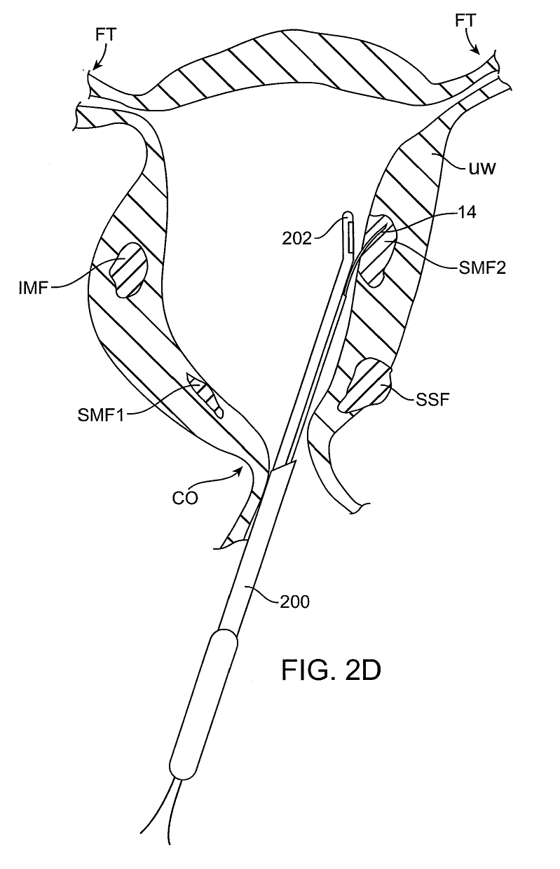

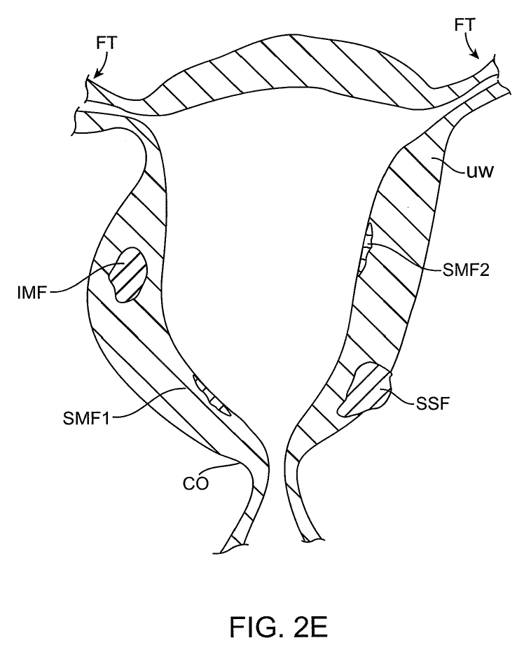

Referring now to FIGS. 2A-2E, use of the visualization and ablation system 200 for selectively ablating submucosal uterine fibroids in accordance with the principles of the present invention will be described. The uterus U of a patient to be treated by the methods of the present invention will frequently include a plurality of fibroids, such as submucosal fibroids SMF 1 and SMF 2, intramural fibroids IMF, and subserosal fibroids SSF, as shown in FIG. 2A. In accordance with the present invention, the submucosal fibroids SMF 1 and SMF 2 will be selectively treated as described below.

As shown in FIG. 2B, the visualization and ablation system 200 is manipulated so that a distal end 202 is advanced through the cervical os CO into the uterus U. Once in place inside the uterus, the device 210 can be scanned so that ultrasonic imaging field 204 locates the various fibroids on and within the uterine wall UW. Once the fibroids have been initially scanned and located, the submucosal fibroids SMF 1 and SMF 2 will be distinguished from the other fibroids, including the intramural fibroids IMF and the subserosal fibroids SSF. Once the submucosal fibroids have been identified, they may be sequentially treated, as shown in FIGS. 2C and 2D. For example, the probe of system 200 may first be manipulated so that the distal end 202 is brought near the first submucosal fibroid SMF 1. Once in place, needle 14 may be advanced into the submucosal fibroid SMF 1, as shown in FIG. 2C, and radiofrequency then delivered to the fibroid.

Once the first submucosal fibroid SMF 1 has been treated, the probe of system 200 may be further manipulated so that the distal end 202 is brought adjacent to the second submucosal fibroid SMF 2, as shown in FIG. 2D. Needle 14 is then advanced into the second fibroid, energy applied, and the fibroid ablated.

Once all of the submucosal fibroids have been treated, as shown in FIG. 2E, the submucosal fibroids will be ablated and diminished in size, while the remaining fibroids including the intramural fibroids IMF and the subserosal fibroids SSF may remain without ablation. By targeting the submucosal fibroids only, the patient can be treated for menorrhagia in a reduced time in comparison to treatments where all or most of the uterine fibroids are treated. As many of the other fibroids will have no or minimal symptoms for the patient, selectively targeting only the submucosal fibroids can be a significant advantage in many patients.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for treating menorrhagia, said method comprising:
providing a visualization and ablation instrument comprising a shaft having a distal end, an imaging transducer near the distal end of the shaft, and an energy delivery element near the distal end of the shaft;
advancing the distal end of the shaft into a uterus;
visualizing fibroids with the imaging transducer while the distal end remains in the uterus;
distinguishing submucosal fibroids from all other fibroids;
selectively delivering energy from the energy delivery element to the submucosal fibroid(s).

2. A method as in claim 1, wherein selectively delivering energy comprises targeting only the submucosal fibroid(s).

3. A method as in claim 1, wherein selectively delivering energy comprises delivering energy only to the submucosal fibroid(s).

4. A method as in claim 1, wherein advancing comprises advancing the distal end through a vagina and cervical os into the uterus.

5. A method as in claim 1, further comprising manipulating a proximal end of the shaft to position the imaging transducer to visualize different wall regions of the uterus.

6. A method as in claim 1, wherein visualizing comprises ultrasonic imaging with an ultrasonic transducer.

7. A method as in claim 1, wherein distinguishing further comprises determining which submucosal fibroids cause bleeding.

8. A method as in claim 1, wherein selectively delivering energy comprises advancing a needle from the shaft into the submucosal fibroid(s) and delivering radiofrequency energy via the needle to the submucosal fibroid(s).

9. A method for treating menorrhagia in a patient, said method comprising:
providing a visualization means including an imaging transducer for visualizing fibroids;
providing an energy delivery element;
employing said visualization means for visualizing uterine fibroids;
visualizing fibroids with the imaging transducer;
distinguishing submucosal fibroids from all other fibroids; and
selectively targeting the submucosal fibroid(s) by delivering energy from the energy delivery element to the submucosal fibroid(s).

10. A method as in claim 9, further comprising inserting said energy delivery element into a uterus of the patient.

11. A method as in claim 10, further comprising inserting said visualization means into the uterus.

12. A method as in claim 9, wherein said energy delivery element comprises a mechanical element.

13. A method as in claim 9, wherein said energy delivery element comprises an ablative element using ablative energy.

14. A method as in claim 9, wherein said energy delivery element and said visualization means are disposed at the distal end of a common shaft.

15. A method as in claim 14, further comprising advancing the distal end of the shaft into a uterus of the patient.

16. A method as in claim 10, wherein said imaging transducer is external of the uterus.

17. A method as in claim 9, wherein said energy from said delivery element is delivered to the fibroid from a position external of a uterus of the patient.

18. A method as in claim 17, wherein said imaging transducer is external of the uterus.

19. A method as in claim 9, further comprising inserting said visualization means into the uterus.

20. A method as in claim 19, wherein said energy from said delivery element is delivered to the fibroid from a position external of a uterus of the patient.
